(19) United States
(12) Patent Application Publication
(10) Pub. No.: US 2019/0246920 A1
(43) Pub. Date: Aug. 15, 2019

(54) INTELLIGENT EXAMINATION DEVICE

(71) Applicant: Han-Wei Zhang, New Taipei City (TW)

(72) Inventor: Han-Wei Zhang, New Taipei City (TW)

(21) Appl. No.: 16/274,761

(22) Filed: Feb. 13, 2019

(30) Foreign Application Priority Data
Feb. 14, 2018 (CN) 201820258903.3

(51) Int. Cl. A61B 5/0215; A61B 5/024; H04N 13/254; H04N 13/207

(52) U.S. Cl. CPC A61B 5/02154; H04N 13/207; H04N 13/254; A61B 5/02416

(57) ABSTRACT

An intelligent examination device includes a processor, an electrode apparatus, and a stereoscopic image capturing apparatus. The electrode apparatus is connected to the processor and includes first and second electrodes. When a detection loop is between the first electrode and the second electrode, measurement information is transmitted to the processor. The stereoscopic image capturing apparatus transmits a biological appearance stereoscopic image to the processor. The processor obtains biological appearance information according to the biological appearance stereoscopic image, and calculates physiological information according to the biological appearance information and the measurement information. The intelligent examination device uses the collaboration of the electrode apparatus and the stereoscopic image capturing apparatus and can achieve the effect of multiple examination devices by a simple mechanism. Therefore, the device costs of medical units and the examination time for a subject can be reduced, and an alert can be provided in real time for underlying symptoms.

### CROSS-REFERENCE TO RELATED APPLICATION

This non-provisional application claims priority under 35 U.S.C. § 119(a) to Patent Application No. 201820258903.3 filed in China, P.R.C. on Feb. 14, 2018, the entire contents of which are hereby incorporated by reference.

### BACKGROUND

### Technical Field

The present disclosure relates to the field of health examination, and in particular, to an intelligent examination device.

### Related Art

In order to perform physical examinations on subjects, medical units such as hospitals and clinics need to purchase various devices for examining different parts of the body and employ medical professionals to conduct the examinations, leading to high manpower and device costs. However, currently many physical examinations are for school, work, passport, and insurance, and purchased devices cannot be fully used. Therefore, depreciation puts heavy burden on medical units.

A subject needs to schedule time to visit a hospital or clinic to undergo an examination. Generally, it takes about half a day to complete the examination, but in fact, the subject spends much time in queuing up for various items of the examination. In addition, after the examination is completed, the subject needs to make an appointment with a doctor for diagnosis based on the examination result. This process is time-consuming and not cost-effective, and also makes the subject less willing to undergo the examination, making many symptoms less likely to be diagnosed early.

### SUMMARY

To solve the problems of the conventional technologies, the present disclosure provides an intelligent examination device. The intelligent examination device includes a processor, an electrode apparatus, and a stereoscopic image capturing apparatus. The electrode apparatus is electrically connected to the processor. The electrode apparatus includes a first electrode and a second electrode. When a detection loop is formed between the first electrode and the second electrode, measurement information is generated and transmitted to the processor. The stereoscopic image capturing apparatus is electrically connected to the processor. The stereoscopic image capturing apparatus captures and transmits a biological appearance stereoscopic image to the processor. The processor obtains biological appearance information according to the biological appearance stereoscopic image, and calculates physiological information according to the biological appearance information and the measurement information.

In some embodiments, the intelligent examination device further includes a light source apparatus, wherein the light source apparatus generates light, the stereoscopic image capturing apparatus captures a feedback image from a position at which the light illuminates and transmits the feedback image to the processor, and the processor further calculates feedback information according to the feedback image, and calculates the physiological information based on the feedback information, the measurement information, and the biological appearance information.

In some embodiments, the intelligent examination device further includes a housing, wherein the processor is mounted inside the housing, and the electrode apparatus and the stereoscopic image capturing apparatus are mounted on the housing. Further, the electrode apparatus is mounted on a surface of the housing.

In some embodiments, the housing may be a wearable object. The processor is disposed inside the housing, and the electrode apparatus and the stereoscopic image capturing apparatus are retractably assembled to the housing. Further, the wearable object is a vest, a jacket, a hat, or a combination thereof.

In some embodiments, the intelligent examination device further includes a communication apparatus. The communication apparatus is electrically connected to the processor, to receive and send out the physiological information.

In some embodiments, the intelligent examination device further includes a first communication apparatus and a second communication apparatus. The first communication apparatus, the processor, and the stereoscopic image capturing apparatus constitute an intelligent processing apparatus, the second communication apparatus and the electrode apparatus constitute a measurement apparatus, and the intelligent processing apparatus and the measurement apparatus are communicatively connected to each other through the first communication apparatus and the second communication apparatus.

Further, the intelligent processing apparatus is a smart phone, a tablet computer, the vehicle, or a combination thereof. In addition, the measurement apparatus is a body fat meter, an electrocardiography measurement unit, an electroencephalography measurement unit, or a combination thereof.

In some embodiments, the stereoscopic image capturing apparatus at least includes two camera lenses. Each of the camera lenses captures an image and transmits the image to the processor. The processor combines the images to generate the biological appearance stereoscopic image.

In some embodiments, the stereoscopic image capturing apparatus further includes an infrared camera unit. The infrared camera unit captures and transmits a temperature distribution image to the processor. The processor calculates temperature feedback information based on the temperature distribution image, and calculates the physiological information based on the temperature feedback information, the measurement information, and the biological appearance information

In some embodiments, the electrode apparatus further includes a plurality of pairs of electrode pads, the pairs of electrode pads are respectively electrically connected to the processor, and when each of the pairs of electrode pads forms a detection loop, monitoring information is generated and transmitted to the processor. The processor calculates the physiological information according to the biological appearance information, the measurement information, and the monitoring information.

In the foregoing embodiments, the intelligent examination device uses the electrode apparatus and the stereoscopic image capturing apparatus to jointly assist in judging the physiological information, and can achieve the effect of multiple examination devices by means of a simple mechanism. Therefore, the device costs of medical units and the examination time for a subject can be reduced, and an alert can be provided in real time for underlying symptoms or possible acute symptoms. An application may also be used to send a notification, to facilitate early diagnosis and early treatment.

### BRIEF DESCRIPTION OF THE DRAWINGS

The present invention will become more fully understood from the detailed description given herein below for illustration only, and thus are not limitative of the present invention, and wherein:

FIG. 1 is a schematic block diagram of an intelligent examination device;

FIG. 2 is a schematic three-dimensional diagram of an intelligent examination device according to a first embodiment;

FIG. 3 is a schematic three-dimensional diagram of an intelligent examination device according to a second embodiment;

FIG. 4 is a schematic three-dimensional diagram of an intelligent examination device according to a third embodiment;

FIG. 5 is a schematic three-dimensional diagram of an intelligent examination device according to a fourth embodiment;

FIG. 6 is a schematic three-dimensional diagram of an intelligent examination device according to a fifth embodiment; and

FIG. 7 is a schematic block diagram of an intelligent examination device according to another embodiment.

### DETAILED DESCRIPTION

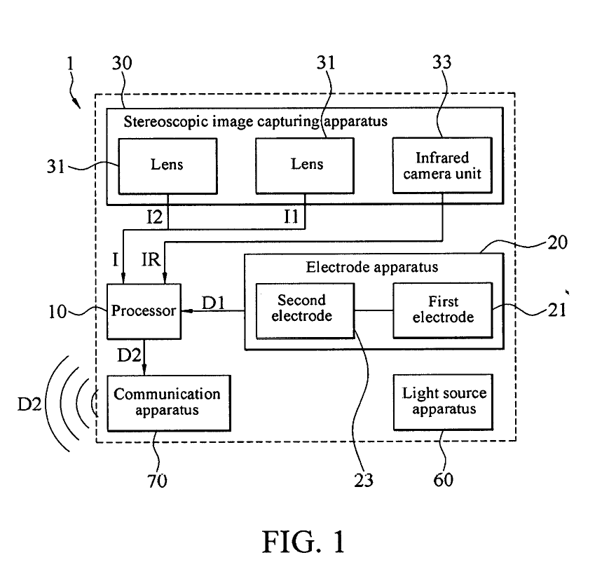

FIG. 1 is a schematic block diagram of an intelligent examination device. As shown in FIG. 1, the intelligent examination device 1 includes a processor 10, an electrode apparatus 20, and a stereoscopic image capturing apparatus 30. The processor 10 is electrically connected between the electrode apparatus 20 and the stereoscopic image capturing apparatus 30.

The electrode apparatus 20 includes a first electrode 21 and a second electrode 23, when the first electrode 21 and the second electrode 23 are respectively in contact with a human body, the first electrode 21 and the second electrode 23 form a detection loop through conduction of the human body. The first electrode 21 and the second electrode 23 generate measurement information D1 through the detection loop. Then, the electrode apparatus 20 transmits the measurement information D1 to the processor 10.

The stereoscopic image capturing apparatus 30 captures and transmits a biological appearance stereoscopic image I to the processor 10. The processor 10 can perform calculation according to the biological appearance stereoscopic image I and convert the biological appearance stereoscopic image I into biological appearance information, and calculate physiological information D2 according to the measurement information D1 and the biological appearance information.

Herein, the measurement information D1 may be a voltage signal or current signal corresponding to bio-electrical resistance. In other words, after receiving the voltage signal or current signal corresponding to bio-electrical resistance, the processor 10 can generate the measurement information D1 according to the voltage signal or current signal. In an embodiment, the measurement information D1 may be a numerical value, but the present disclosure is not limited thereto.

The biological appearance stereoscopic image I is a three-dimensional stereoscopic image corresponding to a subject, and may be a single three-dimensional stereoscopic image or a plurality of continuous three-dimensional stereoscopic images.

The biological appearance information is a numerical value calculated by the processor 10 according to the corresponding biological appearance stereoscopic image I.

The physiological information D2 may be physiological condition information or potential risk information. Therefore, when the subject undergoes an examination through the intelligent examination device 1, measurement information can be obtained through measurement using the detection loop formed between the first electrode 21 and the second electrode 23, and based on the measurement information and the biological appearance stereoscopic image acquired by the stereoscopic image capturing apparatus 30, the physiological information D2 can be quickly obtained. That is to say, with the assistance of the biological appearance stereoscopic image I, the intelligent examination device 1 can diagnose the human body more accurately and further send information about possible symptoms of the body of the subject, thereby solving the prior-art problem that local underlying symptoms cannot be diagnosed based on the measurement information D1.

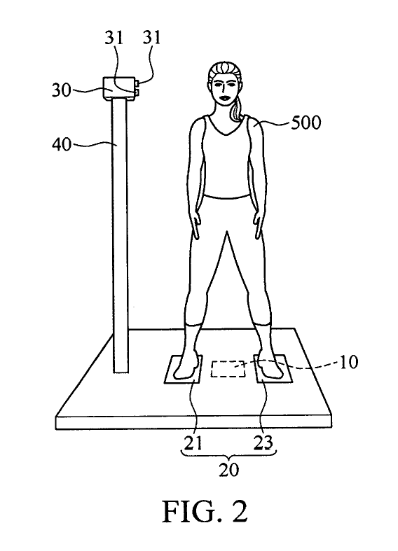

Various implementations of the intelligent examination device 1 will be described below through different embodiments. FIG. 2 is a schematic three-dimensional diagram of an intelligent examination device according to a first embodiment. As shown in FIG. 2, in addition to the processor 10, the electrode apparatus 20, and the stereoscopic image capturing apparatus 30, the intelligent examination device 1 further includes a housing 40 and a telescopic pole 45. The processor 10 is mounted inside the housing 40. The first electrode 21 and the second electrode 23 of the electrode apparatus 20 are mounted on a surface of the housing 40, are separated from each other, and are respectively electrically connected to the processor 10. The stereoscopic image capturing apparatus 30 is mounted on the telescopic pole 45, and is electrically connected to the processor 10. The telescopic pole 45 may be assembled to the housing 40. In this way, the telescopic pole 45 can be adjusted to adjust the position and direction of the stereoscopic image capturing apparatus 30 along with the human body 500, so as to capture a suitable biological appearance stereoscopic image I.

In an embodiment, the first electrode 21 and the second electrode 23 may be circuit boards. When two feet of the human body 500 respectively tread on the first electrode 21 and the second electrode 23, the first electrode 21 and the second electrode 23 may be electrically connected to each other through conduction of the human body 500, to form a detection loop, and measurement information D1 is generated according to bio-electrical resistance of the human body 500. The measurement information D1 may be a voltage signal or current signal. The processor 10 receives the measurement information D1, and may calculate, according to the measurement information D1, body composition information such as breathing, heartbeat, pulse, weight, fat mass, fat-free mass, body fat percentage, muscle mass, bone ratio, skeletal muscle mass, muscle mass, visceral fat ratio, water content, water ratio, and protein ratio corresponding to the bio-electrical resistance of the human body 500.

The stereoscopic image capturing apparatus 30 at least includes two lenses 31. In FIG. 2, the two lenses 31 are used as an example, but the present disclosure is not limited thereto. The two lenses 31 photograph the human body to respectively capture images I1 and I2. The stereoscopic image capturing apparatus 30 may be an independent camera apparatus capable of combining the images I1 and I2 into a three-dimensional biological appearance stereoscopic image I. The stereoscopic image capturing apparatus 30 may alternatively be a lens apparatus, which captures and transmits the images I1 and I2 to the processor 10, so that the processor 10 combines the images I1 and I2 into a three-dimensional biological appearance stereoscopic image I. The processor 10 can calculate the biological appearance information according to the biological appearance stereoscopic image I. The biological appearance information may include height, bust, waist circumference, arm circumference, gender, center of gravity, body volume and the like of the human body 500. Herein, the biological appearance stereoscopic image I is not limited to a full-length image of the human body 500, but may also be a half-length image or a local image. In addition, the processor 10 may further receive continuous biological appearance stereoscopic images I, and determine biological appearance information such as gait, walking speed, and joint angle according to corresponding continuous changes in the biological appearance stereoscopic images I. For example, the two lenses 31 may capture a half-length photo of the human body, to obtain a biological appearance image I about half of the human body 500.

Then, the processor 10 performs recognition according to the biological appearance image I to determine various data such as the size and area of the human body 500 in the biological appearance image I. Then, the processor 10 can further derive various biological appearance information such as the height and gender of the human body 500 by using various data of the biological appearance image I. In addition, although the stereoscopic image capturing apparatus 30 in FIG. 2 obtains the biological appearance information such as height when the human body 500 is standing, the human body 500 in fact may also be sitting or lying, and the processor 10 may calculate corresponding biological appearance information according to the biological appearance image I.

Then, the processor 10 may calculate the physiological information D2 according to the measurement information D1, body composition information corresponding to the measurement information D1, and the related numerical value of the biological appearance information. The physiological information D2 may further include physiological condition information such as basal metabolic rate, metabolic age, body mass index (BMI), degree of obesity, muscle assessment, muscle strength, and posture. The physiological information D2 may also be potential risk information of related symptoms calculated based on related thresholds, for example, risk indexes of symptoms such as sarcopenia, obesity, metabolic syndrome, chronic disease, hypertension, and hip closure injury.

For example, the processor 10 may derive information such as the gender, age, and height from the biological appearance information, and may obtain the weight of the subject from the measurement information D1. Therefore, the processor 10 can calculate the physiological information D2, to be specific, the BMI, and risk information of related symptoms according to the biological appearance information and the measurement information D1. Therefore, an examination can be performed on the subject by using the intelligent examination device 1 without requiring any other input action, greatly simplifying the examination process. Other physiological information D2 may also be obtained in a similar way, that is, derived from the biological appearance information and the measurement information D1, and the details will not be described herein again.

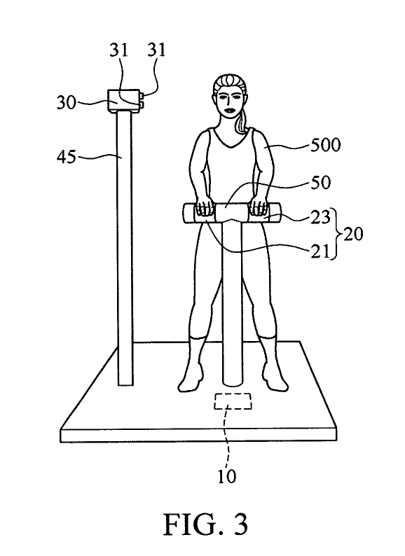

FIG. 3 is a schematic three-dimensional diagram of an intelligent examination device according to a second embodiment. As shown in FIG. 3, the second embodiment is different from the first embodiment in that, the intelligent examination device 1 further includes a handle 50. The first electrode 21 and the second electrode 23 of the electrode apparatus 20 are mounted on a surface of the handle 50. The handle 50 is assembled to the housing 40. When the human body 500 grips the handle 50, the first electrode 21 and the second electrode 23 are electrically connected to each other through conduction of the human body 500, to form a detection loop, and generate measurement information D1. The electrode apparatuses 20 of FIG. 2 and FIG. 3 are merely examples, and the present disclosure is not limited thereto.

The implementations of FIG. 2 and FIG. 3 are applicable to a common physical examination room. Further, the intelligent examination device 1 may be connected to a printer or display (not shown), to print or display the physiological information D2. Therefore, a medical practitioner or the subject can obtain the related physiological information D2 in time. If the medical practitioner determines that potential risk information of a related symptom indicates a high risk or determines that the subject has an acute symptom, the medical practitioner can advise the subject in time to receive a further examination or treatment. In addition, because the costs of the examination device can be reduced, the subject may also purchase an examination device, place the examination device at home for measurement at any time, and track the subject's health status by keeping a long-term record.

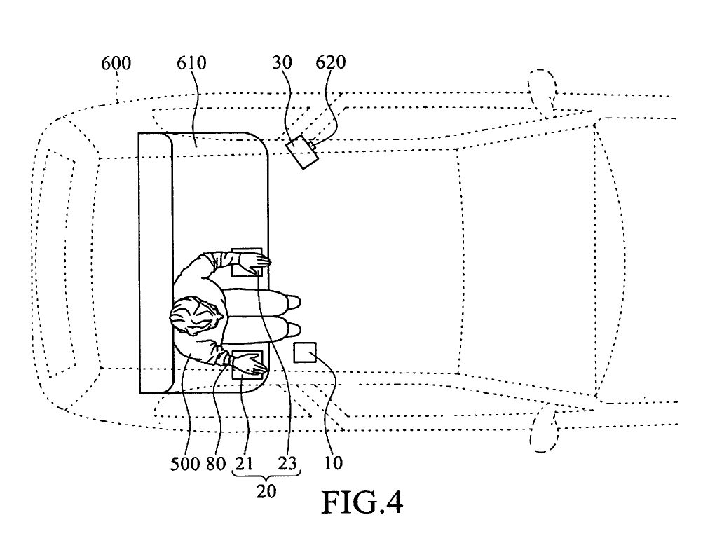

FIG. 4 is a schematic three-dimensional diagram of an intelligent examination device according to a third embodiment. As shown in FIG. 4, the intelligent examination device 1 may be mounted inside a vehicle 600. The processor 10 may be mounted on the vehicle 600 or as part of the vehicle 600. For example, the processor 10 may be an application-specific integrated circuit (ASIC) in a microcomputer of the vehicle 600. The first electrode 21 and the second electrode 23 of the electrode apparatus 20 may be mounted on a seat 610, and the stereoscopic image capturing apparatus 30 may be mounted on a pillar 620. Therefore, when the human body 500 is sitting or driving in the vehicle 600, a physical examination can be performed, thus greatly reducing the examination time. In addition, FIG. 4 is merely an example, but the present disclosure is not limited thereto. The electrode apparatus 20 may be mounted at any position, for example, on the steering wheel or a footpad. The electrode apparatus 20 may also be retractably mounted inside the vehicle 600, and the human body 500 can pull out the electrode apparatus 20 after entering the vehicle, so that the electrode apparatus 20 enters into contact with the human body 500 to form the detection loop. In addition, the stereoscopic image capturing apparatus 30 only needs to be mounted at a position suitable for photographing.

Further, the intelligent examination device 1 may use a communication apparatus related to the vehicle 600 to send the physiological information D2 to a cloud server (not shown) or a smart phone, and may receive alert information. For example, when it is determined that a particular indicator of the physiological information D2 exceeds a criterion and indicates the possibility of an acute symptom, the cloud server sends an alert message to the mobile phone or the vehicle 600 to give an alarm, prompting the subject to see a doctor in time, thereby avoiding a sudden change in physiological condition which may cause traffic accidents.

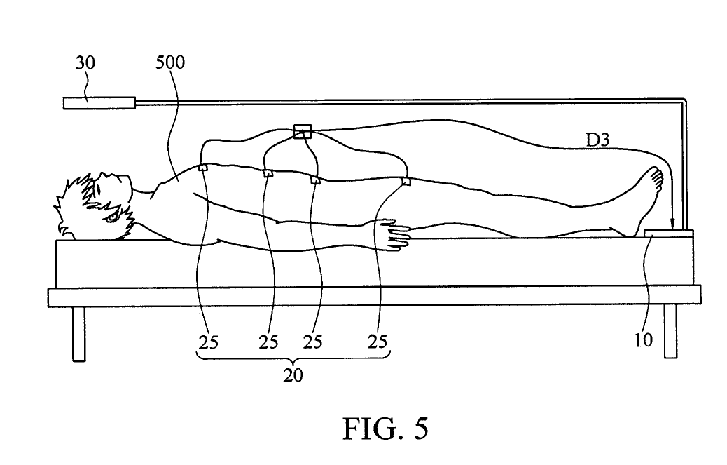

FIG. 5 is a schematic three-dimensional diagram of an intelligent examination device according to a fourth embodiment. As shown in FIG. 5, the electrode apparatus 20 of the intelligent examination device 1 may further include a plurality of pairs of electrode pads 25. The pairs of electrode pads 25 are respectively electrically connected to the processor 10. The electrode pads 25 may be attached to a plurality of areas of the human body 500, for example, attached in pairs to two sides of the shoulder, two arms, two legs, two sides of the back, or two sides of the head, and may form a plurality of detection loops. When the pairs of electrode pads 25 are electrically connected to generate the detection loops, monitoring information D3 is generated and transmitted to the processor 10. The monitoring information D3 may be a potential change value or current change value based on electrocardiographic resistance or electroencephalographic resistance, which may correspond to an electrocardiographic value, an electromyographic value, an electroencephalographic value, and so on, and may be measured with different angles or leads. Further, the intelligent examination device 1 may also be used in combination with a monitoring device (not shown) for a practitioner to view the monitoring information D3 and make judgment.

In addition, the processor 10 of the intelligent examination device 1 may further calculate more physiological information D2, for example, regular heart rhythm, heartbeat status, brain nerve activation status, brain nerve cognitive function status and the like, and risk indexes of dementia, Parkinson's disease and nervous system diseases, based on the measurement information D1, the biological appearance information, and the monitoring information D3. The fourth embodiment provides a simple and fast examination method for medical institutions needing to provide long-term medical care. The intelligent examination device 1 may further be used in combination with a cloud server or mobile phone application, to prompt related underlying symptoms and help the subject to receive a further examination and treatment as soon as possible.

The above manner is merely an example. The intelligent examination device 1 uses only the first electrode 21 and the second electrode 23, and the processor 10 sends different driving signals for control to generate monitoring information D3 corresponding to the electrocardiographic resistance or electroencephalographic resistance. Herein, the electrocardiographic or the electroencephalographic resistance is different from the bio-electrical resistance. Alternatively, the position of contact of the first electrode 21 and the second electrode 23 with the human body 500 may be adjusted, and multiple measurements are performed to obtain the monitoring information D3 corresponding to the electrocardiographic resistance or electroencephalographic resistance.

Referring to FIG. 1 again, the intelligent examination device 1 further includes a light source apparatus 60. The biological appearance image I captured by the stereoscopic image capturing apparatus 30 may also be a local stereoscopic image or continuous stereoscopic images of the human body 500. The lens 31 of the stereoscopic image capturing apparatus 30 may also be a high-power microscopic lens. For example, the light source apparatus 60 may illuminate a finger of the human body 500, and the stereoscopic image capturing apparatus 30 is focused on the finger of the human body 500. Further, the light source apparatus 60 and the stereoscopic image capturing apparatus 30 may be aligned with the position of the finger by using a fixture (not shown). The light source apparatus 60 generates light, for example, light of an infrared wave band, which illuminates a local part of the finger of the human body 500. The stereoscopic image capturing apparatus 30 captures a feedback image of the illuminated finger and transmits the feedback image to the processor 10. The processor 10 analyzes the feedback image and calculates light absorption bands as feedback information. For example, a band that red blood cells carrying oxygen can absorb is 850-1000 nm, and a band that red blood cells not carrying oxygen can absorb is 600-750 nm. Therefore, a ratio of red blood cells carrying oxygen to red blood cells not carrying oxygen may be calculated according to a ratio of the light absorption bands. The processor 10 may further calculate a blood oxygen saturation level according to the feedback information. The processor 10 may further calculate the physiological information D2, for example, hypoxia index, asthma index, cardiopulmonary disease risk index, or chronic obstructive pulmonary disease risk index, according to the feedback information, the measurement information D1, and the biological appearance information.

In some other embodiments, the light source apparatus 60 may also emit light of a particular wave band, to increase the contrast of the feedback image captured by the stereoscopic image capturing apparatus 30, thereby improving the precision of judgment made by the processor 10. For example, with the assistance of the light source apparatus 60, the feedback image captured by the stereoscopic image capturing apparatus 30 under illumination is a stereoscopic image of blood vessels of a particular area, for example, a stereoscopic image of blood vessels of the face or neck. The processor 10 may calculate physiological information D2 such as pulse, heart rate, and cardiopulmonary disease risk index based on the photographing time and continuous changes in the positions and volumes of blood vessels in the feedback image. However, the above description is merely an example, but the present disclosure is not limited thereto.

For example, the particular light source apparatus 60 is used to illuminate blood vessels of a particular part of the human body 500 to increase the contrast. The stereoscopic image capturing apparatus 30 captures a plurality of feedback images. The processor 10 may calculates feedback information such as expansion or shrinkage of blood vessels based on continuous changes of the blood vessels in the plurality of feedback images. The processor 10 may then calculate physiological information D2 such as heart rate, pulse, estimated vascular age, vascular disease risk index according to the pre-captured biological appearance image I, the measurement information D1, and the feedback information. However, the above description is merely an example, but the present disclosure is not limited thereto. In some embodiments, the electrode apparatus 20 (e.g., the first electrode 21, the second electrode 23, or the electrode pad 25) is used to measure pulse of the human body 500 to generate specific pulse graph, e.g., an electrocardiography (ECG). The light source apparatus 60 can generate red-green light. The light source apparatus 60 illuminates the human body 500 with the red-green light. The stereoscopic image capturing apparatus 30 captures a feedback image from a position of the human body 500 illuminated by the red-green light and transmits the feedback image to the processor 10. The processor 10 generates a photoplethysmography (PPG) according to the electrocardiography and the feedback image. In addition, the processor 10 further calculates the physiological information D2 such as blood oxygen saturation (SpO2), bold pressure, condition of cardiopulmonary and cardiovascular disease, and condition of pain according to the photoplethysmography. In some embodiment, the intelligent examination device 1 can further comprise a pressure measurement apparatus 80 (as shown in FIG. 4). For example, the pressure measurement apparatus 80 may be, but not limited to, a known electronic sphygmomanometer. The pressure measurement apparatus 80 may be disposed on a certain position of the seat 610 to facilitate a connection of the human body 500 and the pressure measurement apparatus 80. For instance, the pressure measurement apparatus 80 may be fastened to a wrist and/or an ankle of the human body 500. The pressure measurement apparatus 80 can be used to measure an arterial pressure pulse of the human body 500 such as ankle brachial pressure index (ABI) and cardio-ankle vascular index (CAVI) and transmit the arterial pressure pulse to the processor 10. The processor 10 can calculate the physiological information D2 such as systolic pressure, diastolic blood pressure, pulse, condition of valves of heart, condition of three major vessels of heart, condition of cardiomegaly, and level of arterial stiffness according to the electrocardiography and the arterial pressure pulse. In addition, the physiological information D2 can not only be used to estimate cardiac condition in all dimensions, but can also be used to estimate cardiovascular disease of entire human body.

Referring to FIG. 1 again, the stereoscopic image capturing apparatus 30 further includes an infrared camera unit 33. The infrared camera unit 33 generates and transmits a temperature distribution image IR to the processor 10. The processor 10 calculates temperature feedback information based on the temperature distribution image IR, and calculates the physiological information D2 based on the temperature feedback information, the measurement information D1, and the biological appearance information. In this embodiment, the feedback information may correspond to temperature values of a plurality of parts of the human body 500. Therefore, the intelligent examination device 1 can use the infrared camera unit 33 to assist in determining whether the human body 500 has a fever or suffers from local inflammation. The infrared camera unit 33 may be an independent unit or may be implemented as a filter attached on the lens 31. The above description is merely an example, but the present disclosure is not limited thereto. In some embodiments, the infrared camera unit 33 is a near infrared spectroscopy (NIRS), which is used to perform an optical examination of infrared spectrum. For instance, the infrared camera unit 33 can capture an infrared spectral image of cerebral and neurovascular activities and transmit the infrared spectral image to the processor 10. In addition, the monitoring information D3 further comprises an electroencephalographic value. The processor 10 calculates the physiological information D2 according to the infrared spectral image of cerebral and neurovascular activities and the electroencephalographic value. In such case, the physiological information D2 can be used to exam condition of cerebral neuron activation and condition of neurovascular coupling and can also be used to exam hemoglobin (Hb), oxygenated hemoglobin (HbO₂), oxyhemoglobin (HbO), and deoxyhemoglobin (HbR). In addition, the physiological information D2 can be used to quantify and estimate condition of cerebral cognition function and to quantify dementia, Parkinson's disease, or varied kinds of mental disorder.

In addition, in some embodiments, the intelligent examination device 1 further includes a communication apparatus 70. The communication apparatus 70 is electrically connected to the processor 10, to receive and send out the physiological information D2. The communication apparatus 70 may be a wireless or wired signal transceiver, for example, USB port, Micro USB port, HDMI port, WiFi signal transceiver, microwave signal transceiver, Bluetooth signal transceiver, ZeeBee signal transceiver, or the like. Taking the WiFi signal transceiver for example, the communication apparatus 70 may transmit in real time the physiological information D2 calculated by the processor 10 to a workstation server or cloud server (not shown), for a related medical practitioner or program to make a preliminary judgment. For example, after a physical examination is completed, all the physiological information D2 may be transmitted from the cloud server to a practitioner's computer, so that the practitioner can make a judgment directly based on the physiological information D2 during diagnosis, thereby reducing the examination time for the medical practitioner and the subject.

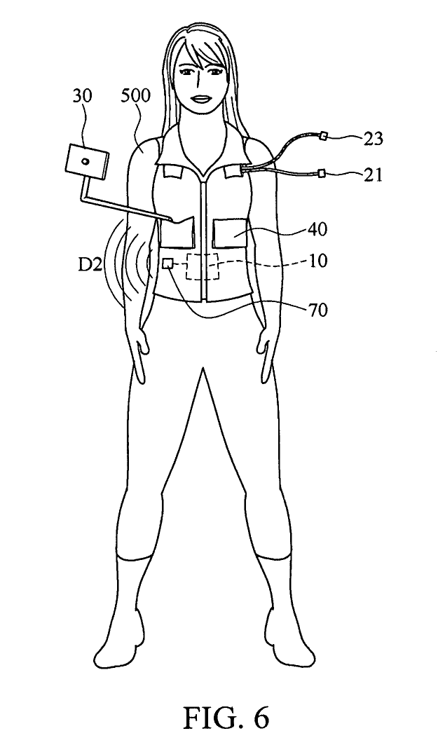

FIG. 6 is a schematic three-dimensional diagram of an intelligent examination device according to a fifth embodiment. As shown in FIG. 6, the housing 40 of the intelligent examination device 1 may be a vest-type wearable object, that is, the housing 40 may be soft or flexible. The processor 10 may be embedded or buried in the housing 40. In addition, the electrode apparatus 20 and the stereoscopic image capturing apparatus 30 are retractably assembled to the housing 40. For example, the electrode apparatus 20 and the stereoscopic image capturing apparatus 30 are connected to the housing 40 in a wired manner. When not in use, the electrode apparatus 20 and the stereoscopic image capturing apparatus 30 may be received inside the housing 40, for example, a pocket. When measurement and photographing need to be performed, the electrode apparatus 20 and the stereoscopic image capturing apparatus 30 are pulled out from the housing. Therefore, the housing is convenient to wear for a medical practitioner or a practitioner needing to provide long-term medical care, so that the subject can be examined quickly, and a preliminary judgment can be made by using the obtained physiological information D2.

As shown in FIG. 6, in some other embodiments, the intelligent examination device 1 in which the housing 40 is a wearable object may further include a communication apparatus 70. For example, the processor 10 and the wireless transmission-type communication apparatus 70 are assembled in the vest-like housing 40. Therefore, after the processor 10 calculates the physiological information D2, the communication apparatus 70 can receive the physiological information D2 and quickly upload the physiological information D2 to a workstation server or cloud server (not shown) for recording. The intelligent examination device 1 of this implementation is suitable for use by the medical practitioner or the subject, can reduce the workload of the medical practitioner and the shift time, and can also avoid mistakes caused by improper scheduling of shifts. Herein, the vest is merely an example, and in fact, the housing 40 may also be a jacket, a hat, or other wearable objects.

For example, the practitioner needing to provide long-term medical care may wear the vest-like intelligent examination device 1, and when meeting the subject during ward rounds, pulls out the electrode apparatus 20 and makes the electrode apparatus 20 enter into contact with the subject to obtain measurement information D1, and pulls out the stereoscopic image capturing apparatus 30 to capture a biological appearance image I. Then, the processor 10 can calculate physiological information D2, and sends the physiological information D2 to a workstation server or cloud server (not shown) through the communication apparatus 70. In this way, related medical institutions can track and keep a long-term record of physiological information D2 of a subject requiring long-term rehabilitation or observation, for example, cancer health management index or hip joint rehabilitation index.

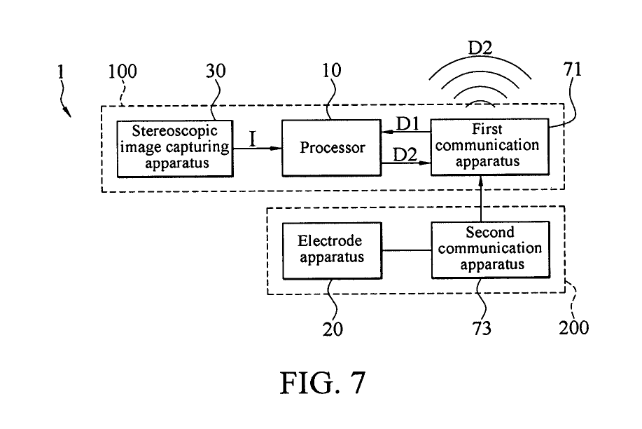

FIG. 7 is a schematic block diagram of an intelligent examination device according to another embodiment. As shown in FIG. 7, the intelligent examination device 1 includes a first communication apparatus 71 and a second communication apparatus 73. The first communication apparatus 71, the processor 10, and the stereoscopic image capturing apparatus 30 constitute an intelligent processing apparatus 100. The second communication apparatus 73 and the electrode apparatus 20 constitute a measurement apparatus 200. The intelligent processing apparatus 100 and the measurement apparatus 200 are communicatively connected to each other through the first communication apparatus 71 and the second communication apparatus 73. The measurement information D1 may be transmitted to the processor 10 through the first communication apparatus 71 and the second communication apparatus 73. The first communication apparatus 71 and the second communication apparatus 73 may be a pair of paired wireless or wired signal transceivers, for example, USB ports, Micro USB ports, HDMI ports, WiFi signal transceivers, microwave signal transceivers, Bluetooth signal transceivers, ZeeBee signal transceivers, or the like.

Further, in some embodiments, the intelligent processing apparatus 100 may be a smart mobile apparatus such as a smart phone or a tablet computer, and the stereoscopic image capturing apparatus 30 may be implemented by a lens of a mobile phone or a tablet computer. Therefore, the intelligent examination device 1 can be implemented by using an existing apparatus, thereby saving the costs required for purchasing related devices, and can provide personalized, private, and customized functions.

Further, the intelligent processing apparatus 100 may also transmit the physiological information D2 to a workstation server or cloud server (not shown) through the first communication apparatus 71, and may also receive an alert message from the workstation server or the cloud server. For example, the workstation server or the cloud server may determine whether there is a sign of an acute disease based on whether a particular numerical value of the physiological information D2 exceeds a criterion, and if yes, sends an alert to the intelligent processing apparatus 100. However, this is merely an example, but the present disclosure is not limited thereto. A notification may also be sent to the subject or the medical practitioner by using an SMS message or voice, to prompt the subject to see a doctor and receive treatment as soon as possible.

In addition, with reference to the foregoing embodiments, the intelligent processing apparatus 100 may also be a vehicle 600. The measurement apparatus 200 may be a wearable object, a fingerprint sensor, a body fat meter, an electrocardiography measurement unit, or an electroencephalography measurement unit, and may also be a vehicle 600. For example, taking FIG. 4 for example, the subject is in the vehicle 600, a smart phone is used as the intelligent processing apparatus 100, and the electrode apparatus 200 disposed in the vehicle 600 and the communication apparatus of the vehicle 600 are used as the measurement apparatus 200. Alternatively, a microcomputer and the communication apparatus of the vehicle 600 and a lens assembled in the vehicle 600 may be used as the intelligent processing apparatus 100, and a body fat meter having a communication apparatus 70a is used as the measurement apparatus 200. In addition, taking FIG. 6 for example, a smart phone may be used as the intelligent processing apparatus 100, and a vest having the electrode apparatus 20 and the communication apparatus 70 may be used as the measurement apparatus 200.

For example, the subject may use a mobile phone as the intelligent processing apparatus 100, and control the stereoscopic image capturing apparatus 30 of the mobile phone to capture a biological appearance image I which is a full-length or half-length photo. In addition, two hands of the subject are in contact with a first electrode 21 and a second electrode 23 of the body fat meter having the wireless communication apparatus 70a. The first electrode 21 and the second electrode 23 faun a detection loop and generate measurement information D1, and then send the measurement information D1 to the mobile phone. The processor 10 of the mobile phone receives the measurement information D1, calculates biological appearance information such as gender, height, and weight, according to the biological appearance image I. Then, the processor 10 of the mobile phone calculates body composition information according to the measurement information D1, and then calculates physiological information D2 such as BMI, muscle mass, bone ratio, and potential disease risk according to the body composition information and the biological appearance information. The above description is merely an example, but the present disclosure is not limited thereto.

In another embodiment, the first electrode 21 may also be disposed on the mobile phone, and one hand of the subject is in contact with the first electrode 21. Then, the subject stands on the second electrode 23 (or attaches the second electrode 23 to a hand or foot), so that the first electrode 21 and the second electrode 23 can form the detection loop. Whereby, the processor 10 can further calculate the physiological information D2 of the subject.

As described in the foregoing embodiments, the intelligent examination device 1 uses the electrode apparatus 20 and the stereoscopic image capturing apparatus 30 to jointly assist in judging the physiological information D2, and can achieve the effect of multiple examination devices by means of a simple mechanism. Therefore, the device costs of medical units and the examination time for a subject can be reduced. Further, a cloud server, an application, and the like may further be used to preliminarily analyze symptoms, so as to provide an alert in real time for underlying symptoms or possible acute symptoms, thereby facilitating early diagnosis and early treatment of the subject.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. An intelligent examination device, comprising:
a processor;
an electrode apparatus, electrically connected to the processor, and comprising a first electrode and a second electrode, wherein when a detection loop is formed between the first electrode and the second electrode, measurement information is generated and transmitted to the processor; and
a stereoscopic image capturing apparatus, electrically connected to the processor, wherein the stereoscopic image capturing apparatus captures and transmits a biological appearance stereoscopic image to the processor, and the processor obtains biological appearance information according to the biological appearance stereoscopic image, and calculates physiological information according to the biological appearance information and the measurement information.

2. The intelligent examination device according to claim 1, further comprising a light source apparatus, wherein the light source apparatus generates light, the stereoscopic image capturing apparatus captures a feedback image from a position at which the light illuminates and transmits the feedback image to the processor, and the processor further calculates feedback information according to the feedback image, and calculates the physiological information based on the feedback information, the measurement information, and the biological appearance information.

3. The intelligent examination device according to claim 2, wherein the light is red-green light, the measurement information comprises an electrocardiography, the processor further generates a photoplethysmography according to the feedback image and the electrocardiography, and the processor further calculates the physiological information according to the photoplethysmography.

4. The intelligent examination device according to claim 1, further comprising a pressure measurement apparatus, wherein the pressure measurement apparatus generates an arterial pressure pulse, the measurement information comprises an electrocardiography, and the processor further generates the physiological information according to the electrocardiography and the arterial pressure pulse.

5. The intelligent examination device according to claim 1, further comprising a housing, wherein the processor is mounted inside the housing, and the electrode apparatus and the stereoscopic image capturing apparatus are mounted on the housing.

6. The intelligent examination device according to claim 5, wherein the electrode apparatus is mounted on a surface of the housing.

7. The intelligent examination device according to claim 5, wherein the housing is a wearable object, the processor is disposed inside the housing, and the electrode apparatus and the stereoscopic image capturing apparatus are retractably assembled to the housing.

8. The intelligent examination device according to claim 7, wherein the wearable object is a vest, a jacket, a hat, or a combination thereof.

9. The intelligent examination device according to claim 1, further comprising a communication apparatus, wherein the communication apparatus is electrically connected to the processor, to receive and send out the physiological information.

10. The intelligent examination device according to claim 1, further comprising a first communication apparatus and a second communication apparatus, wherein the first communication apparatus, the processor, and the stereoscopic image capturing apparatus constitute an intelligent processing apparatus, the second communication apparatus and the electrode apparatus constitute a measurement apparatus, and the intelligent processing apparatus and the measurement apparatus are communicatively connected to each other through the first communication apparatus and the second communication apparatus.

11. The intelligent examination device according to claim 10, wherein the intelligent processing apparatus is a smart phone, a tablet computer, a vehicle, or a combination thereof.

12. The intelligent examination device according to claim 10, wherein the measurement apparatus is a body fat meter, a wearable object, a fingerprint sensor, a vehicle, an electrocardiography measurement unit, an electroencephalography measurement unit, or a combination thereof.

13. The intelligent examination device according to claim 1, wherein the stereoscopic image capturing apparatus at least comprises two camera lenses, each of the camera lenses captures an image and transmits the image to the processor, and the processor combines the images to generate the biological appearance stereoscopic image.

14. The intelligent examination device according to claim 1, wherein the stereoscopic image capturing apparatus further comprises an infrared camera unit, the infrared camera unit captures and transmits a temperature distribution image to the processor, and the processor calculates temperature feedback information based on the temperature distribution image, and calculates the physiological information based on the temperature feedback information, the measurement information, and the biological appearance information.

15. The intelligent examination device according to claim 1, wherein the electrode apparatus further comprises a plurality of pairs of electrode pads, the pairs of electrode pads are respectively electrically connected to the processor, and when each of the pairs of electrode pads forms a detection loop, monitoring information is generated and transmitted to the processor, and the processor calculates the physiological information according to the biological appearance information, the measurement information, and the monitoring information.

16. The intelligent examination device according to claim 15, wherein the stereoscopic image capturing apparatus further comprises an infrared camera unit, the infrared camera unit captures and transmits an infrared spectral image to the processor, the monitoring information comprises an electroencephalographic value, and the processor further calculates the physiological information according to the infrared spectral image and the electroencephalographic value.
